(19) United States
(12) Patent Application Publication
(10) Pub. No.: US 2021/0030348 A1
(43) Pub. Date: Feb. 4, 2021

(54) SYSTEMS AND METHODS TO IMPROVE A USER'S MENTAL STATE

(71) Applicant: Maestro Games, SPC, West Hollywood, CA (US)

(72) Inventors: Yael SWERDLOW, Los Angeles, CA (US); David SHAPENDONK, Los Angeles, CA (US)

(21) Appl. No.: 16/932,550

(22) Filed: Jul. 17, 2020

Related U.S. Application Data
(60) Provisional application No. 62/881,812, filed on Aug. 1, 2019.

(51) Int. Cl. A61B 5/16; G16H 20/70; G06F 3/01

(52) U.S. Cl. CPC A61B 5/165; G06F 3/017; G16H 20/70

(57) ABSTRACT

Systems and methods to improve a user's mental state are disclosed. The method includes playing a segment of a musical performance to a user. The method also includes detecting one or more arm movements of the user, the one or more movements corresponding to movements conducting the musical performance. In response to detecting the one or more movements, the method includes determining a current mental state of the user, and obtaining one or more changes to one or more musical elements of the musical performance that improve the current mental state of the user. The method also includes applying the one or more changes to the one or more musical elements to the segment of the musical performance, and playing the segment of the musical performance to the user.

### CROSS-REFERENCE TO RELATED APPLICATIONS

The present Application claims priority to and benefit of U.S. Provisional Patent Application No. 62/881,812, filed Aug 1, 2019, the disclosure of which is incorporated herein by reference in its entirety.

### BACKGROUND

The present disclosure relates generally to systems and methods to improve a user's mental state.

Certain individuals suffer from temporary or permanently debilitating conditions, such as, but not limited to, anxiety, depression, schizophrenia, Alzheimer's, post-traumatic stress disorder, as well as other types of adverse mental or physical conditions (collectively referred to as “adverse conditions”). Music has sometimes been used to improve the mental state of individuals affected by adverse conditions as well as the mental state of individuals who are not suffering from any adverse condition.

### BRIEF DESCRIPTION OF THE DRAWINGS

Illustrative embodiments of the present invention are described in detail below with reference to the attached drawing figures, which are incorporated by reference herein, and wherein:

FIG. 1 is a network environment for improving a user's mental state in accordance with one embodiment.

FIG. 2 is a system diagram of a system to improve the user's mental state.

FIG. 3 is a system diagram of the backend system of FIG. 1.

FIG. 4 is a flow chart that illustrates a process to improve a user's mental state in accordance with one embodiment.

FIG. 5 is a flow chart that illustrates a process to improve a user's mental state in accordance with another embodiment.

The illustrated figures are only exemplary and are not intended to assert or imply any limitation with regard to the environment, architecture, design, or process in which different embodiments may be implemented.

### DETAILED DESCRIPTION

In the following detailed description of the illustrative embodiments, reference is made to the accompanying drawings that form a part hereof. These embodiments are described in sufficient detail to enable those skilled in the art to practice the invention, and it is understood that other embodiments may be utilized and that logical structural, mechanical, and electrical changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative embodiments is defined only by the appended claims.

The present disclosure relates to systems and methods to improve a user's mental state. As referred to herein, a user's mental state refers to the user's state of mind, or the user's current state of mind. Further, and as referred to herein, a musical performance refers to any audible performance by a solo artist or an ensemble. Examples of musical performances include, but are not limited to, performances by an orchestra, a band, a choir, a section of an orchestra (e.g., strings, woodwinds, brass instruments), a member of the orchestra (e.g., the concertmaster), a lead vocal, or audio performances by other solo or group acts.

The user, while listening to a musical performance, may take on the role of a virtual conductor to interact and change various musical elements of the musical performance. As referred to herein, a musical element is any element of the musical performance that changes audio or visual aspects of the performance. Examples of musical elements include, but are not limited to, tempo, volume, dynamics, cuing certain performers (e.g., for the concertmaster to begin, for the woodwinds to stop playing), as well as other elements that affect audio or visual aspects of the musical performance. In some embodiments, the user utilizes a conducting device (e.g., an electronic device operable to determine a location or orientation of the respective electronic device) to conduct the musical performance. In one or more of such embodiments, the movements of the conducting device are analyzed to determine the user's desired changes to musical elements of the musical performance. In some embodiments, movements of the user's arms are analyzed to determine the user's desired changes to musical elements of the musical performance.

In some embodiments, a visual display of the musical performance is also provided to the user to provide the user with visual interactions with the musical performance. Examples of a visual display of the musical performance include, but are not limited to, members of the musical performance, the performance vista (interior of a concert hall, outside in a forest, ocean, mountain range, or outer space), audience, lighting, special effects, as well as other visual aspects of the musical performance. In one or more of such embodiments, the user selects aspects of the visual display the user would like to view. For example, the user selects whether to view or not view the audience, performers (a specific performer or a group of performers), lighting, special effects, forum, and other aspects of the visual display. In one or more of such embodiments, selection of various aspects of the virtual display are predetermined or are determined based on prior user selections/experience. In one or more of such embodiments, the musical performance takes place at various virtual vistas or points of interests with or without other aspects of the visual display described herein. For example, the musical performance takes place in front of the Eiffel Tower, the Victoria Harbor, the Sydney Opera House, the Burj Khalifa, the Great Wall of China, and the Pyramid of Giza, a natural scene such as the Alaskan mountain range, Yellowstone National Park, and El Capitan, a historical point of interest such as the Hanging Gardens of Babylon, the Colossus of Rhodes, the Lighthouse of Alexandria, and the Temple of Artemis, undersea, outer space, or another point of interest. In one or more of such embodiments, the user selects the virtual vista. In some or more of such embodiments, the virtual vista is predetermined or selected based on prior user selections/experience. In one or more embodiments, the user designs and customizes various aspects of the virtual display. For example, the user customizes the virtual display to include the Pyramid of Giza next to the Eiffel Tower and in front of the Alaskan mountain range for a more pleasant experience. In one or more of such embodiments, the user views the musical performance through a virtual reality headgear. In one or more of such embodiments, the user views the musical performance through an electronic display. Additional descriptions of visual displays of musical performances are provided in the paragraphs below.

While the user performs the role of a virtual conductor, sensors on or proximate to the user measure one or more physical, biological, or neurological measurements of the user to determine the user's current state of mind and how the user's current state of mind is affected by the musical performance (e.g., the audio and visual aspects of the musical performance). Examples of sensors include, but are not limited to, facial recognition sensors, heart rate sensors, movement sensors, blood pressure sensors, oxygen level sensors, digital scales, nano-sensors, body temperature sensors, perspiration detectors, brain wave sensors, as well as other sensors operable to detect physical, biological, or neurological measurements of the user. For example, the user may enjoy the performance of the concertmaster, may smile while listening to the concertmaster' s performance, and may motion the concertmaster to play louder. In one or more embodiments, a facial recognition sensor detects the user's smile as well as other facial expressions of the user while the user interacts with the concertmaster. In another example, where visual aspects of the musical performance are provided to the user, lighting above the orchestra may cause the user discomfort. The user may place a hand between the user's eyes and a screen displaying visual aspects of the musical performance or remove a virtual reality headgear displaying visual aspects of the musical performance to shield the user's eyes from such discomfort. In one or more of such embodiments, one or more sensors detect the user's hand movements to shield the user's eyes as well as other physical, biological, or neurological expressions of discomfort.

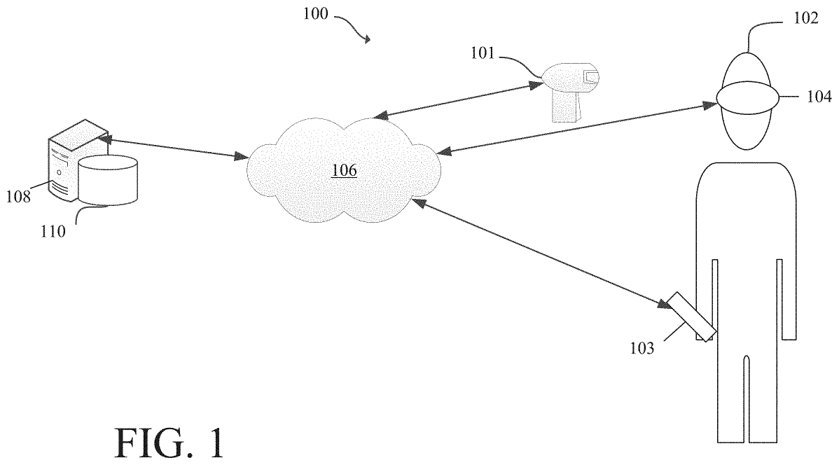

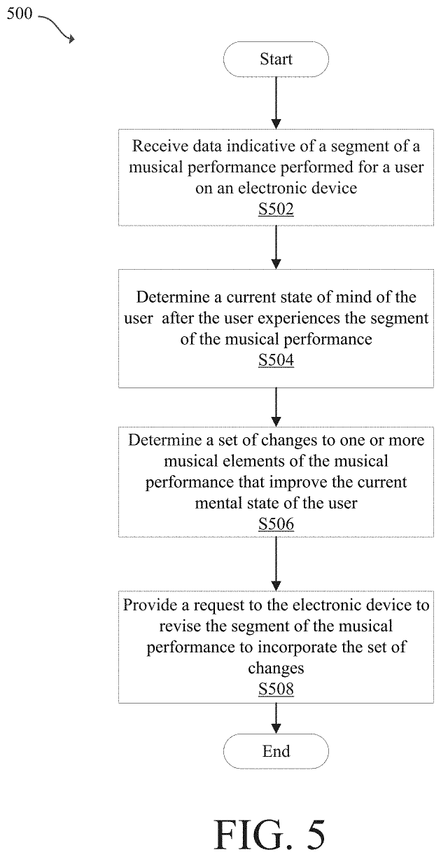

Data indicative of the positive physical, biological, or neurological expressions (such as the user smiling when listening to the concertmaster's performance) and negative physical, biological, or neurological expressions (such as the user shielding the user's eyes from light above the orchestra) are aggregated and are analyzed to determine which musical elements (audio and visual) are positively received by the user, which musical elements are negatively received by the user, and which musical elements have little or no effect on the user. In some embodiments, a backend system illustrated in FIG. 1 aggregates prior biological and neurological expressions of the user in response to interacting with musical performances, and categorizes musical elements that cause positive, neutral, or negative reactions from the user. In one or more of such embodiments, the system also analyzes user experiences from other users (e.g., users within a general population, users sharing similar physical, biological, or neurological characteristics), and estimates which musical elements would cause positive, neutral, or negative reactions from the user based on reactions of other users. Additional descriptions of systems and methods for making such determinations are provided in the paragraphs below and are illustrated in at least FIG. 5.

Further, a determination of changes to existing musical elements are made, such as by the backend system of the previous example. For example, where the backend system determines that the lighting is causing the user discomfort, the backend system may request the visual display (e.g., the virtual reality headgear) to reduce the intensity of the lighting. Similarly, where the backend system determines that the user enjoys the performance of the concertmaster, the backend system requests an audio device playing the audio of musical performance (which, in some embodiments, is a component of the visual display) to increase the volume of the concertmaster. In some embodiments, the backend system may request the audio device to play a different segment of the musical performance, commence a new musical performance, as well as make other changes to musical elements of the musical performance to improve the user's mental state while listening to the musical performance. Similarly, the backend system may also request the visual display to change various visual elements of the musical performance to improve the user's mental state while the user visualizes the musical performance.

Although the foregoing paragraphs describe a single-user experience, in some embodiments, the systems described herein also allow multiple users to simultaneously engage and participate in a musical performance. In one or more of such embodiments, different users participate in different aspects of the musical performance, e.g., one user conducts the strings, another user conducts the windpipes, and a third user conducts the vocals. In one or more of such embodiments, users take turns conducting the musical performance. For example, each of three users takes turn conducting while the other two users observe visual aspects of the musical performance while waiting for their respective turn to conduct the musical performance. In one or more of such embodiments, the users receive conducting scores for their respective performances to engage in friendly conducting battles. In one or more of such embodiments, musical and visual aspects of the musical performance are uploadable by the user (with the user's consent) to a social media platform or to another location on the Internet. In one or more embodiments, the systems described herein scores each user's performance based on a set of criteria, and dynamically provides each user with their respective score during a musical performance. In one or more of such embodiments, the systems described herein compare each user's conducting to the tempo of the musical performance the user is conducting and awards the respective user points based on how in-sync the respective user's movement is relative to the tempo. In one or more of such embodiments, where faster/quieter musical performances are associated with shorter and/or quicker arm movements, each user is awarded points based on how close the respective user's arm movements are to a predefined set of movements that correspond to directing the musical performance. Similarly, where loud volumes of musical performances are associated with more expansive arm movements, each user is awarded points based on how close the respective user's arm movements are to a predefined set of movements that correspond to directing the musical performance. In one or more embodiments, where a musical performance contains a crescendo that is associated with a pause, or other changes in tempo or volume, each user is awarded points based on how close the respective user's arm movements are to a predefined set of movements that correspond to conducting the musical performance during the crescendo, or other changes in tempo or volume. In one or more of such embodiments, criteria for scoring a user's performance is predetermined. In one or more of such embodiments, criteria for scoring a user's performance is adjustable by the respective user, by a group of users engaged in a multiplayer musical performance, or by a third party. In one or more embodiments, user scores are provided to all of the users that are engaged in a multiplayer session. In one or more of such embodiments, a user has an option not to view the scores or one or more components of the scores of one or more users engaged in the multiplayer session. In one or more embodiments, the system also analyzes feedback (such as, but not limited to, physical, biological, or neurological measurements) of the users that are engaged in multiplayer musical performances, and performs a comparative analysis of the feedback. Additional descriptions of systems of methods to improve the user's mental state are provided in the paragraphs below and are illustrated in at least FIGS. 1-5.

Now turning to the figures, FIG. 1 is a network environment 100 for improving a user's mental state in accordance with one embodiment. Network environment 100 includes a visual device 104 placed over the eyes of a user 102. As referred to herein, user 102 includes any individual who experiences one or more musical performances. Although in some embodiments, user 102 experiences one or more adverse conditions, in other embodiments, user 102 does not suffer from any adverse condition. Visual device 104 includes any electronic device operable to display one or more visual elements of the musical performance (e.g., the performances, the audience, the forum of the musical performance, the lighting, as well as other visual aspects of the musical performance). Although FIG. 1 illustrates the visual device 104 as a virtual reality headgear, the visual device 104 may also be implemented as a display screen, tablet computer, smartphone, laptop computer, desktop computer, smart television, electronic watch, PDA, as well as similar electronic devices having hardware, software, and/or firmware that are operable to display or project one or more visual elements of the musical performance.

In the embodiment of FIG. 1, user 102 is holding a conducting device 103. As referred to herein, a conducting device 103 is a device the user waves around when conducting music. In the embodiment of FIG. 1 conducting device 103 is a controller. Additional examples of conducting device 103 include, but are not limited to, smartphones, smart watches, tablet computers, electronic accessories (e.g., electronic pens), as well as non-electronic apparatuses the user may wave around to conduct music. In some embodiments, conducting device 103 is operable of detecting the user's hand/arm movement, and determining a conducting gesture based on the user's hand/arm movement. In some embodiments, where conducting device 103 is not an electronic device, another electronic device placed nearby (such as sensor 101 or a different sensor or device (not shown)) detects movements of conducting device 103, and determines musical interpretations of user 102 based on movements of conducting device 103.

In the embodiment of FIG. 1, audio of musical performances is played by conducting device 103. As audio of a musical performance is played and visual elements of the musical performance are displayed on visual device 104, user 102 motions conducting device 103 to conduct the musical performance and to change various musical and visual elements of the musical performance in accordance to interpretations of user 102. For example, visual depictions of an ensemble performing on a stage at the Sydney Opera House are displayed on visual device 104 while Symphony No. 9 is playing from a speaker of conducting device 103. As user 102 visualizes and hears Symphony No. 9, user 102 may perform certain motions with conducting device 103 to adjust certain musical elements of Symphony No. 9. For example, user 102 may direct members of the chorus to sing louder, the strings to speed up the tempo, the woodwinds to play softer, and to make other adjustments to the musical elements of the musical performance. Similarly, user 102 may also make adjustments to visual elements of the musical performance, such as, but not limited to, requesting stage hands to adjust the lighting, requesting the audience to be quiet at the start of the musical performance, requesting the musical performers to stand and bow to the audience and the audience applauds the performance, as well as adjustments to other visual elements of the musical performance.

One or more sensors are placed proximate to user 102 to monitor one or more physical, biological, or neurological measurements of the user while user 102 conducts musical performances. In the embodiment of FIG. 1, a sensor 101 is placed on or near user 102 to obtain one or more physical, biological, or neurological measurements of user 102. In the embodiment of FIG. 1, sensor 101 is a facial expression scanner. Additional examples of sensor 101 include, but are not limited to, voice recognition devices, heart rate sensors, movement sensors, blood pressure sensors, oxygen level sensors, digital scales, nano-sensors, body temperature sensors, perspiration detectors, brain wave sensors, as well as other sensors operable to detect physical, biological, or neurological measurements of user 102.

In the embodiment of FIG. 1, sensor 101 continuously or periodically scans facial expressions of user 102 while user 102 listens to and interacts with a musical performance. Continuing with the foregoing example, where user 102 is listening to Symphony No. 9, sensor 101 continuously or periodically captures facial expressions of user 102 as user 102 conducts Symphony No. 9. In some embodiments, where sensor 101 is a movement sensor, sensor 101 continuously measures arm/hand movements of user 102 as user 102 conducts Symphony No. 9 or another musical performance. In one or more of such embodiments, sensor 101 detects different gestures made by user 102 that correspond to instructions to members of an ensemble performing a musical performance, instructions to stage crew, instructions to audiences, or other instructions a conductor of a musical performance may provide by moving the conductor's baton or through hand movements. In some embodiments, where sensor 101 is an audio detector or a video recorder, sensor 101 detects words or other audio feedback of user 102. In one or more of such embodiments, where user 102 utters “what an amazing voice” after hearing the voice of a soprano singer, and utters “too loud” after hearing the performance by a string quartet, words and other audio and video feedback of user 102 are detected by sensor 101. The audio and video feedback of user 102 are then dynamically or periodically transmitted through a network 106, to a backend system 108.

Network 106 can include, for example, any one or more of a cellular network, a satellite network, a personal area network (PAN), a local area network (LAN), a wide area network (WAN), a broadband network (BBN), a RFID network, a Bluetooth network, a device-to-device network, the Internet, and the like. Further, the network 106 can include, but is not limited to, any one or more of the following network topologies, including a bus network, a star network, a ring network, a mesh network, a star-bus network, tree or hierarchical network, or similar network architecture. The network 106 may be implemented using different protocols of the internet protocol suite such as TCP/IP. The network 106 includes one or more interfaces for data transfer. In some embodiments, the network 106 includes a wired or wireless networking device (not shown) operable to facilitate one or more types of wired and wireless communication between sensor 101, conducting device 103, visual device 104, backend system 108, and other electronic devices (not shown) communicatively connected to the network 106. Examples of the networking device include, but are not limited to, wired and wireless routers, wired and wireless modems, access points, as well as other types of suitable networking devices described herein. Examples of wired and wireless communication include, Ethernet, WiFi, Cellular, LTE, GPS, Bluetooth, RFID, as well as other types of communication modes described herein.

As referred to herein, a backend system 108 is any electronic device or system operable to determine a user's current state of mind, such as the state of mind of a user such as user 102 after the user perceives a musical performance or a segment of the musical performance, and determine one or more changes to one or more musical elements of the musical performance that improves the current mental state of the user. For example, where user 102 smiles after the beginning of a performance by a soprano, and data indicative of a change in facial expression of user 102 is provided to backend system 108, backend system 108 determines that gradually increasing the volume of the soprano's voice and displaying the soprano's lyrics would improve the current state of mind of user 102. Similarly, where user 102 winces after a change (e.g., a user initiated change) to speed up the tempo of a musical performance, and a sudden increase in the heart rate of user 102 is detected, backend system 108 determines that lowering the volume of the musical performance and slowing down the tempo of the musical performance would improve the current state of mind of user 102. In the embodiment illustrated in FIG. 1, backend system 108 is a server system. Additional examples of backend systems include, but are not limited to, desktop computers, laptop computers, tablet computers, and other devices and systems operable to determine the current state of mind of a user and determine one or more changes to musical elements of a musical performance to improve the user's current state of mind. In some embodiments, backend system 108 is hosted at a remote location relative to the location of user 102. In other embodiments, backend system 108 is a system that is local relative to the location of user 102.

In some embodiments, backend system 108 determines which changes to musical elements of a musical performance would improve the current mental state of user 102 based on prior data associated with user 102. For example, where backend system 108 determines that user 102 has just completed conducting the second movement of Symphony No. 6, backend system 108 analyzes prior responses of user 102 to Symphony No. 6 or similar musical performances. In one or more of such embodiments, where backend system 108 determines that user 102 has conducted Symphony No. 6 three times within the last week (month, year, or another threshold period of time), and each time, user 102 conducted the third movement in Allegretto instead of the default Allegro tempo, backend system 102 determines that user 102 would prefer the tempo of the third movement to be Allegretto instead of the default Allegro. In accordance with another example, where backend system 108 determines that user 102 became sad after hearing the fourth movement of Symphony No. 6, backend system 108 determines that the mental state of user 102 would improve if a different musical performance is presented to user 102 after user 102 conducts the third movement of Symphony No. 6. In some embodiments, backend system 108 assigns different weights to different prior responses of user 102. In one or more of such embodiments, prior responses of user 102 obtained more than a threshold period of time ago (e.g., a year, a month, a week, or another period of time) is assigned a first weight and prior responses of user 102 obtained less than or equal to the threshold period of time ago is assigned a second weight. In some embodiments, backend system 108 also assigns different weights based on the relevance of prior responses of user 102.

In some embodiments, backend system 108 analyzes not only the prior responses of user 102, but also prior responses of other users, and determines which changes to musical elements of a musical performance would improve the current mental state of user 102 based on aggregated user responses from multiple users. In one or more of such embodiments, backend system 108 analyzes all user data aggregated within a threshold period of time (e.g., within a year, a month, a week, a day, or another period of time). In one or more of such embodiments, backend system 108 analyzes relevant users, such as family members, friends of the user, users within the same age group of user 102, users suffering from the same adverse condition as user 102, or based on other categories that include user 102. For example, where user 102 suffers from post-traumatic stress disorder, and backend system 108 determines that 95% of other users who suffer from post-traumatic stress disorder responded positively when Symphony No. 6 is played below a first threshold decibel, and 80% of users who suffer from post-traumatic stress disorder responded negatively when Symphony No. 6 is played a second threshold decibel, backend system 108 also determines that when user 102 desires to conduct Symphony No. 6, changing the default volume of Symphony No. 6 to below the first threshold decibel would improve the mental state of user 102, whereas changing the default volume of Symphony No. 6 would cause the mental state of user 102 to deteriorate.

Backend system 108 includes or is communicatively connected to a storage medium 110 that contains aggregated user data. The storage medium 110 may be formed from data storage components such as, but not limited to, read-only memory (ROM), random access memory (RAM), flash memory, magnetic hard drives, solid state hard drives, CD-ROM drives, DVD drives, floppy disk drives, as well as other types of data storage components and devices. In some embodiments, the storage medium 110 includes multiple data storage devices. In further embodiments, the multiple data storage devices may be physically stored at different locations. In one of such embodiments, the data storage devices are components of a server station, such as a cloud server. In another one of such embodiments, the data storage devices are components of a local management station of a facility user 102 is staying. As referred to herein, aggregated user data include prior data indicative of user selections of musical performances, user interactions with musical performances (e.g., how the user conducts musical performances), user responses to certain musical or visual elements of musical performances, (including, but not limited to physical, biological, neurological, and other measurable user responses), prior user preferences (e.g., genre of musical performance, tempo of musical performance, volume of musical performance, as well as other measurable user preferences), changes to musical or visual elements that improved the user's state of mind, changes to musical or visual elements that caused a deterioration of the user's state of mind, as well as other measurable data of user 102 obtained from sensor 101, conducting device 103, visual device 104, as well as other sensors/devices (not shown) operable to measure data of user 102 and transmit the measured data of user 102 to backend system 108.

In some embodiments, aggregated data also includes user medical records, including, but not limited to adverse conditions of user 102 and other users, as well as histories of treatments of user 102 and other users, and user responses to such treatments. In some embodiments, aggregated data also includes data of other users who have engaged in one or more conducting sessions. In some embodiments, aggregated data also includes data indicative of calibrations of sensors and devices used to measure user 102, default settings of such sensors and devices, and user-preferred settings of such sensors and devices. In some embodiments, storage medium 110 also includes instructions to receive data indicative of a segment of a musical performance played to a user, such as user 102, instructions to determine a current state of mind of the user after the user perceives the segment of the musical performance, instructions to provide a request to an electronic device (e.g., conducting device 103, visual device 104, or another device (not shown)) to play the revised segment of the musical performance which incorporates the one or more changes, as well as other instructions described herein to improve the user's state of mind.

Backend system 108, after determining musical elements and visual elements of the musical performance that improve the current mental state of user 102, transmits requests to conducting device 103 and visual device 104 to play segment of the musical performance with the one or more changes. For example, after backend system 108 determines that playing Fur Elise at approximately 60 decibels while simultaneously displaying musical notations of Fur Elise improves a state of mind of user 102 (e.g., alleviates an adverse condition of user 102), backend system 108 instructs conducting device 103 to output Fur Elise at approximately 60 decibels and instructs visual device 104 to display musical notations of Fur Elise. In some embodiments, where backend system 108 receives a user instruction (e.g., to increase the volume of Fur Elise to greater than 90 decibels), and determines that user 102 previously reacted negatively to listening to Fur Elise at such volume, backend system 108 instructs conducting device 103 not to increase the volume above a tolerable threshold (e.g., 70 decibels, 75 decibels, 80 decibels, or another threshold).

Conducting device 103 and visual device 104, after receiving instructions from backend system 108 to modify or change musical and visual elements of a musical performance, applies such modifications in the musical performance or a subsequent segment of the musical performance to improve the user's state of mind. Sensor 101, conducting device 103, and visual device 104 continuously or periodically measure user feedback and transmit user feedback via network 106 to backend system 108. User feedback of user 102, as well as other users, are aggregated by backend system 108 and are utilized by backend system 108 to make future recommendations and to modify existing recommendations. As such, as user 102 continues to conduct musical performances, backend system 108 becomes more and more fine-tuned to personal preferences of user 102, and is operable to make personalized changes to musical or visual elements of musical performances that improve the state of mind of user 102.

Although FIG. 1 illustrates conducting device 103 and visual device 104, in some embodiments, operations of conducting device 103 and visual device 104 are performed by a single electronic device. For example, in some embodiments, conducting device 103 is also operable to project visual elements of the musical performance. Further, in some embodiments, a conducting device is not used to conduct musical performances. In one or more of such embodiments, where user 102 is not holding a conducting device 103, motions of an arm of user 102 are used to interpret conducting instructions of user 102. In one or more of such embodiments, sensor 101 captures arm movements of user 102, and backend system 108 determines conducting instructions based on arm movements of user 102. In one or more of such embodiments, visual device 104 provides both audio and visual elements of musical performances to user 102. Further, in some embodiments, only musical elements of musical performances are provided to user 102. In one or more of such embodiments, user 102 does not engage visual device 104. In some embodiments, backend system 108 and devices providing audio and visual elements of musical performances are incorporated into a single device. In one or more of such embodiments, backend system 108 is a component of visual device 104, which also provides audio of musical performances. Further, although FIG. 1 illustrates a single sensor 101, multiple sensors 101 may be placed proximate to user 102 to monitor different physical, biological, and neurological responses of user 102. Further, although FIG. 1 illustrates sensor 101, conducting device 103, and visual device 104 as separate components, in some embodiments, sensor is a built-in component of conducting device 103 or visual device 104.

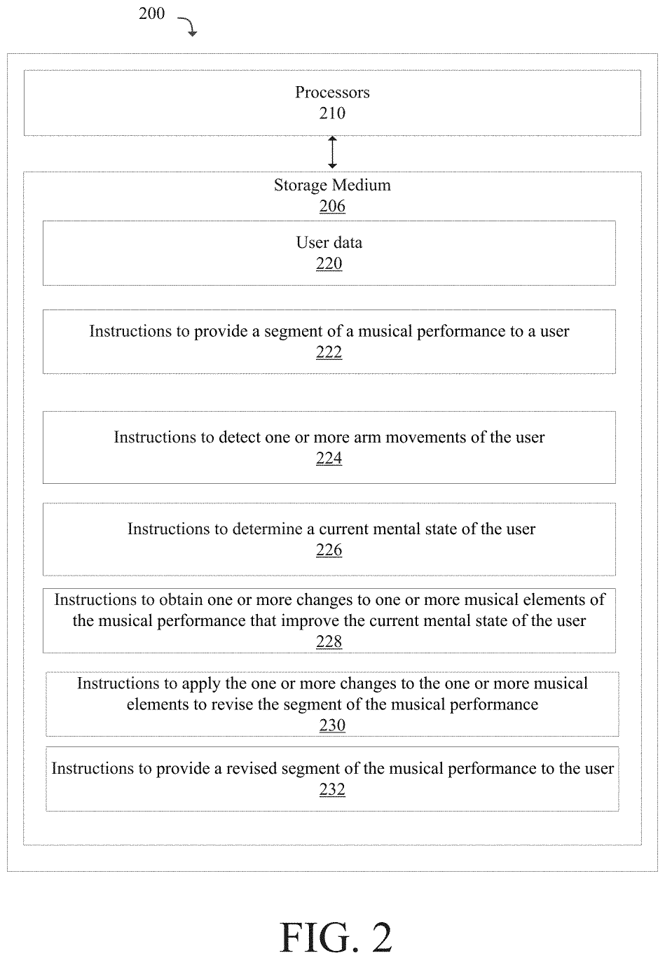

FIG. 2 is a system diagram of a system 200 to improve the user's mental state. System 200 includes a storage medium 206 and processors 210. Storage medium 206 may be formed from data storage components such as, but not limited to, read-only memory (ROM), random access memory (RAM), flash memory, magnetic hard drives, solid-state hard drives, CD-ROM drives, DVD drives, floppy disk drives, as well as other types of data storage components and devices. In some embodiments, storage medium 206 includes multiple data storage devices. In further embodiments, the multiple data storage devices may be physically stored at different locations. User data, such as the user's current state of mind, the user's preferred device settings, as well as other types of data associated with the user, are stored at a first location 220 of storage medium 206. As shown in FIG. 2, instructions to provide a segment of a musical performance to a user are stored at a second location 222 of storage medium 206. Further, instructions to detect one or more arm movements of the user are stored at a third location 224 of the storage medium 206. Further, instructions to determine a current mental state of the user are stored at a fourth location 226 of storage medium 206. Further, instructions to obtain one or more changes to one or more musical elements of the musical performance that improve the current mental state of the user are stored at a fifth location 228 of storage medium 206. Further, instructions to apply the one or more changes to the one or more musical elements to revise the segment of the musical performance are stored at a sixth location 230 of storage medium 206. Further, instructions to provide a revised segment of the musical performance to the user are stored at a seventh location 232 of storage medium 206. In some embodiments, system 200 represents the system of visual device 104, conducting device 103, or backend system 108 of FIG. 1. In some embodiments, system 200 is a standalone system that is communicatively connected to visual device 104, conducting device 103, and backend system 108.

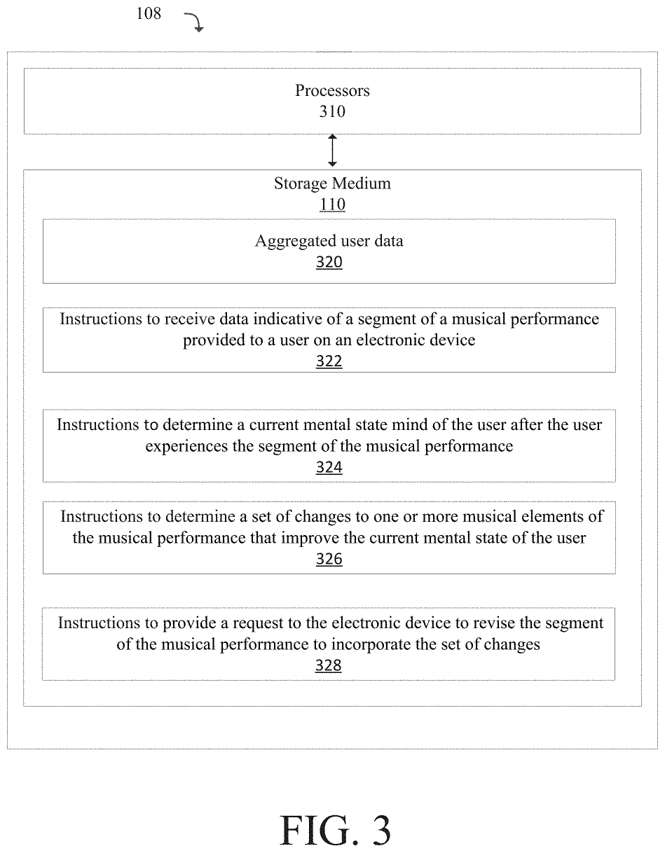

FIG. 3 is a system diagram of backend system 108 of FIG. 1. Backend system 108 includes or is communicatively connected to storage medium 110 and processors 310. Aggregated data, such as the user's performance history, performance histories of other users, as well as other types of data associated with different users, are stored at a first location 320 of storage medium 206. As shown in FIG. 3, instructions to receive data indicative of a segment of a musical performance provided to a user on an electronic device are stored at a second location 322 of storage medium 110. Further, instructions to determine a current state of mind of the user after the user experiences the segment of the musical performance are stored at a third location 324 of the storage medium 110. Further, instructions to determine a set of changes to one or more musical elements of the musical performance that improve the current mental state of the user are stored at a fourth location 326 of storage medium 110. Further, instructions to provide a request to the electronic device to revise the segment of the musical performance to incorporate the set of changes are stored at a fifth location 328 of storage medium 110. In the embodiment of FIG. 1, backend system 108 is communicatively connected to conducting device 103 and visual device 104 via network 106. In some embodiments, backend system 108 is a component of conducting device 103, visual device 104, or another electronic device that the user interacts with or is positioned near the user during a musical performance.

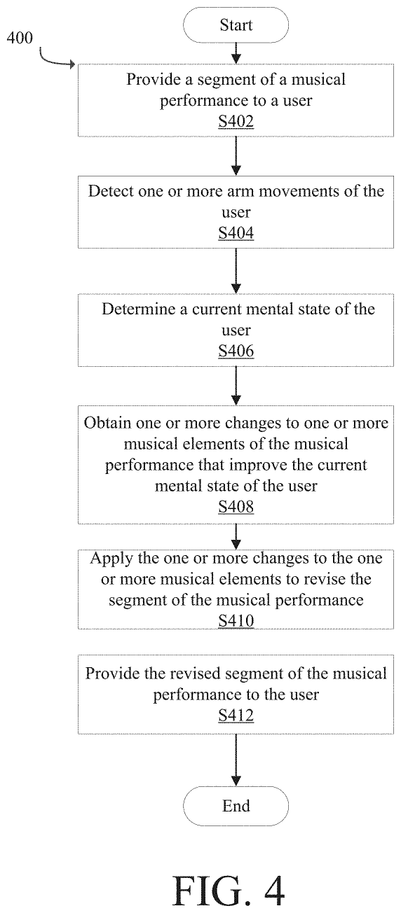

FIG. 4 is a flow chart that illustrates a process to improve a user's mental state in accordance with one embodiment. Although the paragraphs below describe the operations of process 400 being performed by conducting device 103 (such as a processor of conducting device) and visual device 104 (such as a processor of visual device 104) illustrated in FIG. 1, such operations may be performed by only conducting device 103, only visual device 104, or by other devices (not shown) described herein. Further, although operations in the process 400 are shown in a particular order, certain operations may be performed in different orders or at the same time where feasible.

At block S402, a segment of a musical performance is provided to a user, such as user 102 of FIG. 1. The segment of musical performance may be a segment of a solo performance, a duet, a quartet, or an ensemble of musicians. In some embodiments, user 102 selects a specific musical performance user 102 would like to conduct. In some embodiments, user 102 selects a genre (e.g., classical music, rock and roll, opera, or another genre) of music the user would like to conduct. In some embodiments, conducting device 103, visual device 104, backend system 108, or another device or system described herein selects a musical performance for user 102. In one or more of such embodiments, backend system 108 selects a musical performance based on prior selections and feedbacks of user 102. In the embodiment of FIG. 1, visual device 104 also provides user 102 with visual elements of the musical performance (e.g., the performers, the audience, the forum of the musical performance, the lighting of the musical performance, as well as other visual elements user 102 would experience if user 102 is experiencing a live experience).

At block S404, one or more arm movements of user 102 are detected by one or more sensors, such as by sensors of conducting device 103 of FIG. 1. In some embodiments, one or more sensors, such as sensor 101 of FIG. 1 capture arm movements of user 102. In the embodiment of FIG. 1, conducting device 103 operates similar to a baton of a conductor. In one or more of such embodiments, user 102 provides conducting instructions by waving conducting device 103 as if conducting device 103 is a baton. In some embodiments, user 102 conducts musical performances without a conducting device, such as conducting device 103 of FIG. 1. In one or more of such embodiments, arm movements of user 102 are treated as movements of a baton and conducting instructions from user 102 are interpreted (e.g., by visual device 104, backend system 108, or another device or system described herein) based on arm movements of user 102.

At block S406, and in response to detecting one or more arm movements of user 102 or movements of conducting device 103, a determination of a current mental state of user 102 is made. In the embodiment of FIG. 1, sensor 101 scans facial expressions of user 102 to determine the current mental state of user 102. In some embodiments, additional sensors, such as heart rate sensors, movement sensors, blood pressure sensors, oxygen level sensors, digital scales, nano-sensors, body temperature sensors, perspiration detectors, brain wave sensors, as well as other sensors operable to detect physical, biological, or neurological measurements of user 102 are placed proximate to user 102 and are utilized to determine the current state of user 102.

At block S408, one or more changes to musical elements of the musical performance that improve the mental state of the user are obtained. In the embodiment of FIG. 1, backend system 108 determines which changes should be applied and provides proposed changes to conducting device 103 and visual device 104. Additional descriptions of operations performed by backend system 108 or other devices or systems described herein to determine which changes should be applied are illustrated in at least FIG. 5 and are described herein.

At block S410, changes to musical elements are applied to revise the segment of the musical performance. In the embodiment of FIG. 1, conducting device 103 and visual device 104 modify musical and visual elements of the musical performance based on proposed changes communicated by backend system 108. For example, where backend system 108 determines that the volume and light intensity of the musical performance are causing user discomfort, backend system 108 requests conducting device 103 to reduce the volume of the musical performance, and requests visual device 104 to reduce the light intensity.

At block S412, the revised segment of the musical performance is provided to the user. In the embodiment of FIG. 1, the musical performance is revised to include musical and visual elements that benefit the state of mind of user 102. Continuing with the previous example, conducting device 103, in response to receiving a request from backend system 108 to reduce the volume of the musical performance, reduces the volume of the musical performance to a level more suitable for user 102. Similarly, visual device 104, in response to receiving a request from backend system 108 to reduce the intensity of visual elements of the musical performance, also reduces the light intensity to a level more suitable for user 102. As user 102 continues to conduct music, additional physical, biological, and neurological measurements of user 102 are communicated to backend system 108. Backend system 108, in turn, continuously or periodically analyzes measurements of user 102 to determine changes to musical and visual elements that would be beneficial to user 102, and requests conducting device 103 and visual device 104 to apply such changes to the musical performances conducted by user 102.

FIG. 5 is a flow chart that illustrates a process to improve a user's mental state in accordance with one embodiment. Although the paragraphs below describe the operations of process 500 being performed by backend system 108 illustrated in FIG. 1, such operations may also be performed by other devices (not shown) described herein. Further, although operations in the process 500 are shown in a particular order, certain operations may be performed in different orders or at the same time where feasible.

At block S502, data indicative of a segment of a musical performance played to a user on an electronic device are received. In the illustrated embodiment of FIG. 1, backend system 108 receives data indicative of musical performances provided by conducting device 103 and visual device 104. Further, in the illustrated embodiment of FIG. 1, data indicative of physical, biological, and neurological measurements of user 102 are obtained by sensor 101 or other sensors (not shown), and are provided to backend system 108. In some embodiments, backend system 108, after obtaining a user's consent, stores anonymized data of the user's musical performance in storage medium 110. In some embodiments, backend system 108 aggregates data of multiple users based on categories such as, but not limited to, age, occupation, background, and other quantifiable classification standards.

At block S504, a determination of the current state of mind of the user is made after the user experiences the segment of musical performance. In the illustrated embodiment of FIG. 1, backend system 108 determines the current state of mind of user 102 based on data obtained from sensor 101. In some embodiments, conducting device 103 and visual device 104 also contain sensors or components that make physical, biological, and neurological measurements of user 102. In one or more of such embodiments, conducting device 103 and visual device 104 also provide data indicative of measurements of user 102 to backend system 108.

At block S506, a determination of a set of changes to one or more musical elements of the musical performance that improve the current mental state of the user is made. In the illustrated embodiment of FIG. 1, backend system 108 determines changes to musical and visual elements of the musical performance to improve the current mental state of the user. In some embodiments, backend system 108 is pre-programmed (e.g., by an operator, by user 102, or by another individual) to request certain changes to musical and visual elements based on certain responses of user 102. For example, backend system 108, in response to determining that user 102 screamed after listening to a new rock and roll song, determines that user 102 is negatively impacted by the new rock and roll song, and requests conducting device 103 and visual device 104 to provide user 102 with a different song. Further, backend system 108 also determines not to play the same song to user 102 in the future.

In some embodiments, backend system 108 assesses aggregated user data stored in storage medium 110 to determine prior user experiences of user 102 and determines changes to musical and visual elements based on prior user experiences of user 102. In one or more of such embodiments, backend system 108 assigned different weights to different user experiences. For example, backend system 108 assigns a lower weight to prior user experiences experienced more than a first threshold time period ago, and assigns a higher weight to prior user experiences experienced less than a second threshold time period ago. Moreover, backend system 108 determines changes to musical and visual elements in accordance with weights assigned to different prior user experiences of user 102.

In some embodiments, backend system 108 also assesses storage medium 110 for prior user experiences of other users (not shown), and determines changes to musical and visual elements based on prior user experiences of the other users. In one or more of such embodiments, backend system 108 qualifies prior user experiences of other users used to determine proposed changes to the musical and visual elements of musical performances presented to user 102. In one or more of such embodiments, backend system 108 considers only users suffering from identical or similar adverse conditions as user 102. In one or more of such embodiments, backend system 108 only considers users within the same age group as user 102. In one or more of such embodiments, backend system 108 only considers users within the same geographic region as user 102, or shares another quantifiable similarity as user 102. In one or more of such embodiments, backend system 108 assigns different weights to different categories. For example, prior experiences of users who share the same adverse condition as user 102 are assigned a first weight, whereas prior experiences of users who are within the same age group as user 102 are assigned a second weight that is less than the first weight. Additional descriptions of different weight systems applied by backend system 108 when determining whether to make a recommendation based on prior user experiences of user 102 or other users are provided herein.

At block S508, a request to revise the segment of the musical performance to incorporate the set of changes is provided to the electronic device. In the embodiment of FIG. 1, backend system 108 requests conducting device 103 and virtual display 104 to change musical and visual elements of the musical performance determined to improve the state of mind of user 102. Additional descriptions of operations performed by conducting device 103 and virtual display 104 after receiving the request from backend system 108 are illustrated in FIG. 4, and are described herein.

As used in this specification and any claims of this application, the terms “computer”, “server,” “processor,” and “memory,” all refer to electronic or other technological devices. As used in this specification and in any claims of this application, the terms “computer readable medium” and “computer readable media” are entirely restricted to tangible, physical objects that store information in a form that is readable by a computer. These terms exclude any wireless signals, wired download signals, and any other ephemeral signals.

The above-disclosed embodiments have been presented for purposes of illustration and to enable one of ordinary skill in the art to practice the disclosure, but the disclosure is not intended to be exhaustive or limited to the forms disclosed. Many insubstantial modifications and variations will be apparent to those of ordinary skill in the art without departing from the scope and spirit of the disclosure. The scope of the claims is intended to broadly cover the disclosed embodiments and any such modification.

The above disclosed embodiments have been presented for purposes of illustration and to enable one of ordinary skill in the art to practice the disclosed embodiments, but is not intended to be exhaustive or limited to the forms disclosed. Many insubstantial modifications and variations will be apparent to those of ordinary skill in the art without departing from the scope and spirit of the disclosure. For instance, although the flow charts depict a serial process, some of the steps/blocks may be performed in parallel or out of sequence, or combined into a single step/block. The scope of the claims is intended to broadly cover the disclosed embodiments and any such modification. Further, the following clauses represent additional embodiments of the disclosure and should be considered within the scope of the disclosure:

Clause 1, a method to improve a user's mental state, the method comprising: providing a segment of a musical performance to a user; detecting one or more arm movements of the user, the one or more arm movements corresponding to movements conducting the musical performance; in response to detecting the one or more arm movements: determining a current mental state of the user; obtaining one or more changes to one or more musical elements of the musical performance that improve the current mental state of the user; applying the one or more changes to the one or more musical elements to revise the segment of the musical performance; and providing a revised segment of the musical performance to the user.

Clause 2, a method of clause 1, wherein detecting the one or more arm movements of the user comprises detecting one or more movements of a conducting device held in an arm of the user.

Clause 3, the method of clauses 1 or 2, further comprising: providing a visual display of the segment of the musical performance to the user; and in response to detecting the one or more arm movements: obtaining one or more changes to one or more visual elements of the musical performance that improve the current mental state of the user; applying the one or more changes to the one or more visual elements to revise the segment of the musical performance; and providing a visual display of the revised segment of the musical performance to the user.

Clause 4, the method of any of clauses 1-3, wherein providing a visual display comprises providing a visual display of a performance vista of the musical performance, and wherein applying the one or more changes to the one or more visual elements comprises changing the performance vista of the musical performance.

Clause 5, the method of any of clauses 1-4, wherein providing a visual display comprises providing a visual display of a performer of the musical performance, and wherein applying the one or more changes to the one or more visual elements comprises removing the visual display of the performer.

Clause 6, the method of any of clauses 1-5, further comprising determining a similar musical performance that was previously provided to the user; and determining a positive user response to a change made to the similar musical performance, wherein obtaining the one or more changes comprises obtaining the change made to the similar musical performance.

Clause 7, the method of any of clauses 1-6, further comprising monitoring one or more physical signs of the user, wherein determining the current mental state comprises determining the current mental state based on at least one of the one or more physical signs of the user.

Clause 8, the method of any of clauses 1-7, further comprising monitoring one or more biological signs of the user, wherein determining the current mental state comprises determining the current mental state based on at least one of the one or more biological signs of the user.

Clause 9, the method of any of clauses 1-8, further comprising monitoring one or more neurological signs of the user, wherein determining the current mental state comprises determining the current mental state based on at least one of the one or more neurological signs of the user.

Clause 10, the method of any of clauses 1-9, further comprising determining a verbal response of the user, wherein determining the current mental state comprises determining the current mental state based on the verbal response of the user.

Clause 11, the method of any of clauses 1-10, further comprising providing a conducting score of the musical performance to the user.

Clause 12, a system to improve a user's mental state, comprising: an electronic device operable to provide a segment of a musical performance to a user; a sensor operable to detect one or more arm movements of the user, the one or more arm movements corresponding to movements conducting the musical performance; and a processor operable to: determine a current mental state of the user based on the one or more arm movements of the user; obtain one or more changes to one or more musical elements of the musical performance that improve the current mental state of the user; apply the one or more changes to the one or more musical elements to revise the segment of the musical performance; and provide a revised segment of the musical performance to the user.

Clause 13, the system of clause 12, wherein the electronic device is a visual device that is operable to display one or more visual elements of the musical performance.

Clause 14, the system of clause 13, further comprising a conducting device, wherein the sensor is operable to detect movement of the conducting device to determine the one or more arm movements of the user.

Clause 15, a method to improve a user's mental state, the method comprising: receiving data indicative of a segment of a musical performance provided to a user on an electronic device; determining a current mental state mind of the user after the user experiences the segment of the musical performance; determining a set of changes to one or more musical elements of the musical performance that improve the current mental state of the user; and providing a request to the electronic device to revise the segment of the musical performance to incorporate the set of changes.

Clause 16, the method of clause 15, wherein determining the one or more changes to the one or more musical elements of the musical performance comprises: analyzing a plurality of changes to one or more musical elements of one or more previously-provided musical performances provided to one or more users; and selecting one or more of the plurality of changes that improved the mental state of the one or more users.

Clause 17, the method of clause16, further comprising assigning a weight to each of the plurality of changes to the one or more musical elements, wherein selecting the one or more of the plurality of changes comprises selecting the one or more of the plurality of changes based on a weighted value of each of the plurality of changes to the one or more musical elements.

Clause 18, the method of any of clauses 15-17, further comprising analyzing medical records of the user, wherein determining the set of changes to the one or more musical elements is based on the medical records of the user.

Clause 19, the method of any of clauses 15-18, further comprising: receiving data indicative of one or more movements of the user while conducting the musical performance; comparing the one or more movements of the user to a default set of movements to conduct the musical performance; determining a conducting score of the user based on a comparison of the one or more movements of the user to the default set of movements to conduct the musical performance; and providing the conducting score to the electronic device.

Clause 20, the method of any of clauses 15-19, further comprising: providing the segment of the musical performance to a second user that is concurrently conducting the musical performance with the user; detecting one or more arm movements of the second user; in response to detecting the one or more arm movements of the second user: determining a current mental state of the second user; obtaining a second set of changes to one or more musical elements of the musical performance that improve the current mental state of the second user; applying the second set of changes to the one or more musical elements to revise the segment of the musical performance; and providing a revised segment of the musical performance to the second user.

As used herein, the singular forms “a,” “an,” and “the” are intended to include the plural forms as well, unless the context clearly indicates otherwise. It will be further understood that the terms “comprise” and/or “comprising,” when used in this specification and/or the claims, specify the presence of stated features, steps, operations, elements, and/or components, but do not preclude the presence or addition of one or more other features, steps, operations, elements, components, and/or groups thereof In addition, the steps and components described in the above embodiments and figures are merely illustrative and do not imply that any particular step or component is a requirement of a claimed embodiment.

What is claimed is:

1. A method to improve a user's mental state, comprising:
providing a segment of a musical performance to a user;
detecting one or more arm movements of the user, the one or more arm movements corresponding to movements conducting the musical performance;
in response to detecting the one or more arm movements:
determining a current mental state of the user;
obtaining one or more changes to one or more musical elements of the musical performance that improve the current mental state of the user;
applying the one or more changes to the one or more musical elements to revise the segment of the musical performance; and
providing a revised segment of the musical performance to the user.

2. The method of claim 1, wherein detecting the one or more arm movements of the user comprises detecting one or more movements of a conducting device held in an arm of the user.

3. The method of claim 1, further comprising:
providing a visual display of the segment of the musical performance to the user; and
in response to detecting the one or more arm movements:
obtaining one or more changes to one or more visual elements of the musical performance that improve the current mental state of the user;
applying the one or more changes to the one or more visual elements to revise the segment of the musical performance; and
providing a visual display of the revised segment of the musical performance to the user.

4. The method of claim 3, wherein providing a visual display comprises providing a visual display of a performance vista of the musical performance, and wherein applying the one or more changes to the one or more visual elements comprises changing the performance vista of the musical performance.

5. The method of claim 3, wherein providing a visual display comprises providing a visual display of a performer of the musical performance, and wherein applying the one or more changes to the one or more visual elements comprises removing the visual display of the performer.

6. The method of claim 3, further comprising:
determining a similar musical performance that was previously provided to the user; and
determining a positive user response to a change made to the similar musical performance, wherein
obtaining the one or more changes comprises obtaining the change made to the similar musical performance.

7. The method of claim 1, further comprising monitoring one or more physical signs of the user, wherein determining the current mental state comprises determining the current mental state based on at least one of the one or more physical signs of the user.

8. The method of claim 1, further comprising monitoring one or more biological signs of the user, wherein determining the current mental state comprises determining the current mental state based on at least one of the one or more biological signs of the user.

9. The method of claim 1, further comprising monitoring one or more neurological signs of the user, wherein determining the current mental state comprises determining the current mental state based on at least one of the one or more neurological signs of the user.

10. The method of claim 1, further comprising determining a verbal response of the user, wherein determining the current mental state comprises determining the current mental state based on the verbal response of the user.

11. The method of claim 1, further comprising providing a conducting score of the musical performance to the user.

12. A system to improve a user's mental state, comprising:
an electronic device operable to provide a segment of a musical performance to a user;
a sensor operable to detect one or more arm movements of the user, the one or more arm movements corresponding to movements conducting the musical performance; and
a processor operable to:
determine a current mental state of the user based on the one or more arm movements of the user;
obtain one or more changes to one or more musical elements of the musical performance that improve the current mental state of the user;
apply the one or more changes to the one or more musical elements to revise the segment of the musical performance; and
provide a revised segment of the musical performance to the user.

13. The system of claim 12, wherein the electronic device is a visual device that is operable to display one or more visual elements of the musical performance.

14. The system of claim 13, further comprising a conducting device, wherein the sensor is operable to detect movement of the conducting device to determine the one or more arm movements of the user.

15. A method to improve a user's mental state, comprising:
receiving data indicative of a segment of a musical performance provided to a user on an electronic device;
determining a current mental state of the user after the user experiences the segment of the musical performance;
determining a set of changes to one or more musical elements of the musical performance that improve the current mental state of the user; and
providing a request to the electronic device to revise the segment of the musical performance to incorporate the set of changes.

16. The method of claim 15, wherein determining the set of changes to the one or more musical elements of the musical performance comprises:
analyzing a plurality of changes to one or more musical elements of one or more previously-provided musical performances provided to one or more users; and
selecting one or more of the plurality of changes that improved the current mental state of the one or more users.

17. The method of claim 16, further comprising assigning a weight to each of the plurality of changes to the one or more musical elements, wherein selecting the one or more of the plurality of changes comprises selecting the one or more of the plurality of changes based on a weighted value of each of the plurality of changes to the one or more musical elements.

18. The method of claim 15, further comprising analyzing medical records of the user, wherein determining the set of changes to the one or more musical elements is based on the medical records of the user.

19. The method of claim 15, further comprising:
receiving data indicative of one or more movements of the user while conducting the musical performance;
comparing the one or more movements of the user to a default set of movements to conduct the musical performance;
determining a conducting score of the user based on a comparison of the one or more movements of the user to the default set of movements to conduct the musical performance; and
providing the conducting score to the electronic device.

20. The method of claim 15, further comprising:
providing the segment of the musical performance to a second user that is concurrently conducting the musical performance with the user;
detecting one or more arm movements of the second user;
in response to detecting the one or more arm movements of the second user:
determining a current mental state of the second user;
obtaining a second set of changes to one or more musical elements of the musical performance that improve the current mental state of the second user;
applying the second set of changes to the one or more musical elements to revise the segment of the musical performance; and
providing a revised segment of the musical performance to the second user.
